(19) United States
(12) Patent Application Publication
(10) Pub. No.: US 2011/0313338 A1
(43) Pub. Date: Dec. 22, 2011

(54) ANKLE JOINT BANDAGE

(75) Inventors: Ronny Baetz, Vogtlaendisches Oberland (DE); Gerald Stier, Langenwetzendorf (DE); Gerhard Roebelt, Zeulenroda-Triebes (DE)

(73) Assignee: BAUERFEIND AG, Zeulenroda-Triebes (DE)

(21) Appl. No.: 13/146,785

(22) PCT Filed: Jan. 29, 2010
(86) PCT No.: PCT/EP2010/000556

§ 371 Date: Sep. 8, 2011

(30) Foreign Application Priority Data
Jan. 29, 2009 (DE) 10 2009 006 628.4

(51) Int. Cl. A61F 5/00

(52) U.S. Cl. 602/27

(57) ABSTRACT

The invention relates to an ankle joint bandage comprising a stocking (4) running over the middle foot and able joint and a fixing strap enclosing the foot (1) wherein the fixing strap is medially connected to an anchor point (9) on the stocking (4) in the middle foot region. From there the strap (8) may be placed on the sole side around the middle foot crossing the middle of the instep from the medial side to loop around the shin (3) above the ankle, then crossing laterally the middle of the instep again and runs around the middle foot on the sole side again and may be fixed to a bandage region above the sole.

The invention relates to an ankle joint bandage comprising a stocking running over at least the middle foot and the ankle joint, and a fixing strap enclosing the foot.

A bandage of this type is described in DE 43 18 791 A1. This prior art ankle joint bandage includes an elastic stocking which extends over the middle foot and the ankle joint and is used for holding support members in place. Such a support member is a rein-like element made of a tear-resistant and high tensile material that essentially extends over the ankle joint and the heel area on both sides of the respective foot portion in the manner of a stirrup. It furthermore includes an elastic strap which extends medially from and across the heel area on the side of the sole and then crosses the back of the foot, from there loops around the lower leg above the ankle joint and finally ends on the back of the foot where it is fixed to the strap traversing the instep. This design of a prior art ankle joint bandage is aimed at acting on the heel area, and no particular effect on the middle foot portion, in particular a stabilization of the latter, is intended here.

Known from DE 200 05 742 U1 is another ankle joint bandage in which a single strap that is in part elastic and in part inelastic runs laterally from the edge of a foot to the instep of the middle foot and then loops around the lower leg above the ankle. Consequently, as the strap simply just runs across the foot once, it can only stabilize the foot asymmetrically to prevent the user from twisting his ankle in this one direction. This ankle joint bandage therefore does not allow for a comprehensive stabilisation of the foot.

It is the object of the invention to create an ankle joint bandage which above all prevents the foot from going over its ankle diagonally towards the inside and twisting at the same time. This will on the one hand prevent such a dangerous movement and on the other hand give the user, in particular a sportsperson, the impression, through suitable signalling to the nerves, that their foot will not be particularly strained in the respective direction. According to the invention, this is accomplished by medially connecting the fixing strap as an elastic strap to a starting or anchoring point on the stocking in the middle foot region. From there, the strap may be passed around the middle foot on the sole side and then runs across the middle of the instep from the medial side to then loop around the lower leg above the ankle. From there, the strap will laterally cross the middle of the instep again, then run around the middle foot on the sole side once more and may then be fixed to a bandage region above the sole.

Once the strap has been passed around the middle foot on the side of the sole again, it may be applied to the anchoring point region using a hook-and-loop fastener. The strap can also be applied to the foot in such a way that, once it has run around the middle foot on the side of the sole again, it will then continue to the medial side of the strap area which loops around the lower leg above the ankle, where it will then be attached to the strap by means of a hook-and-loop type fastener.

Because the strap loops around the middle foot and part of the lower leg above the ankle, the strap basically extends in the shape of a figure of eight, which will above all prevent any twisting of the foot diagonally to the actual normal direction of the foot. This will support the foot in a particularly stable manner and still enable a certain mobility, as far as the elasticity of the strap allows such. As a result, the user of the bandage will become aware of wrong movements at an early stage when his foot is under a certain strain. What adds to the beneficial effects of this ankle joint bandage design, and especially of the way the fixing strap is passed around the foot, is that the fixing strap, once it has looped around the lower leg, will run around the middle foot from the medial side again and may then be attached to the upper side of the middle foot. Passing the strap around the middle foot once more increases the effect of the fixing strap and thus reduces the danger of injury to the foot due to respective wrong movements. Moreover, passing the fixing strap around the foot twice in the shape of a figure of eight will make it considerably more comfortable when worn.

It is considered advantageous to design the fixing strap in such a way that it consists of an inelastic and an elastic portion, with the elastic portion facing the medial attachment point and the inelastic portion facing the hook-and-loop fastener. Making use of the elastic portion, this design allows the fixing strap to be fitted around the foot closely and with some tension, The additionally applied inelastic portion will then prevent any substantial further movement of the foot by means of this elastic support of the foot with the inelastic portion of the fixing strap. This essentially also makes it easier to apply the bandage, for which purpose the user will first pass the elastic portion around his middle foot and will only continue to apply the inelastic part once the elastic part is in place and feels tight but not unpleasant.

Further advantages, features and potential applications of the present invention may be gathered from the description which follows, in conjunction with the embodiments illustrated in the drawings.

Throughout the description, the claims and the drawings, those terms and associated reference signs will be used as are notable from the enclosed list of reference signs. In the drawings

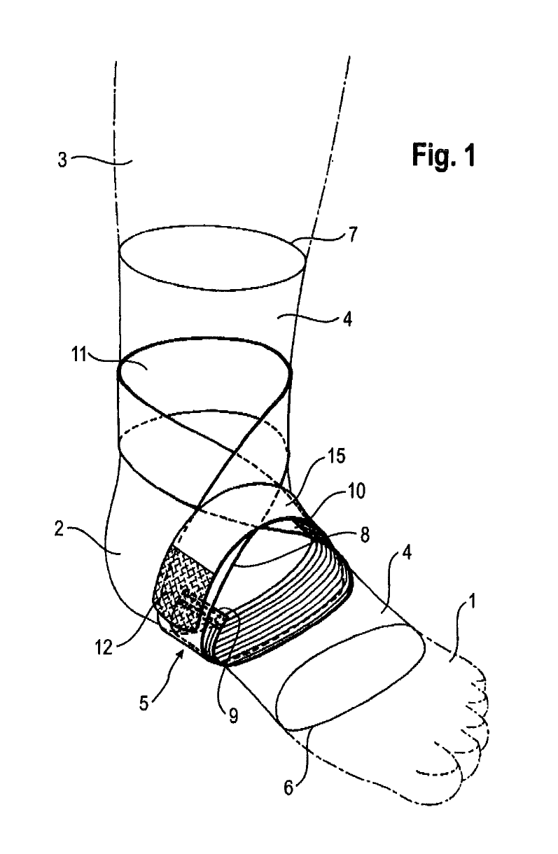

FIG. 1 is a view of the ankle joint bandage in which the strap loops around the middle foot twice and its end is attached to the starting point of the strap, and

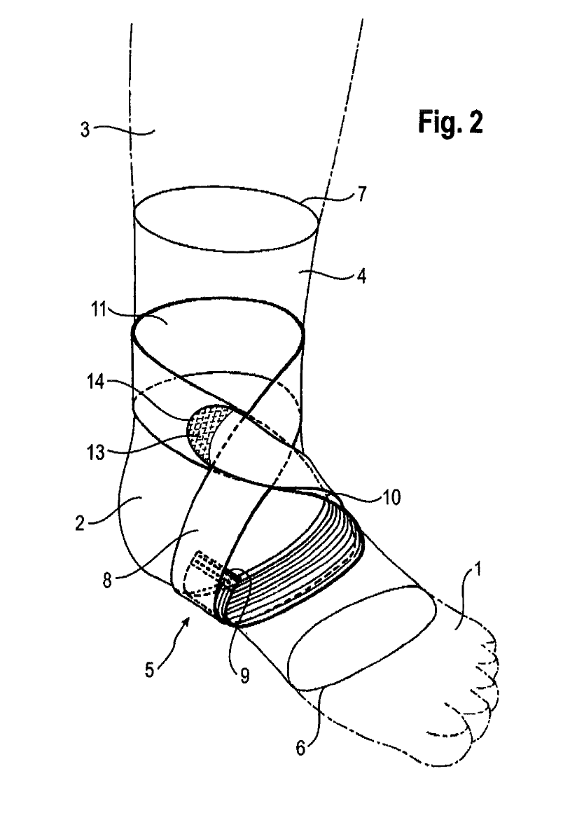

FIG. 2 is a view of the ankle joint bandage similar to that of FIG. 1, in which, however, the end of the strap is attached at a position in the region of the lower leg above the ankle.

FIG. 1 shows a left foot 1 with its heel 2 and part of the lower leg 3. A stocking 4 is pulled over the foot 1, which stocking covers the area of the middle foot 5 and the heel 2 and extends up to the lower leg 3. The end of the stocking 4 that covers the foot is marked 6 and the end of the stocking that covers the lower leg is marked 7.

The ankle joint bandage includes a fixing strap consisting of a strap 8 which has one of its ends connected to the stocking 4 at the anchoring point 9, i.e. medially in the region of the middle foot 5 above the sole. From this anchoring point 9, the strap 8 runs around the sole and then, coming from the side, extends across the middle foot 5 in the region 10 and from there runs around the lower leg 3 (and thus also the stocking 4) in a loop 11. After this loop, the strap 8 then passes across the middle of the instep and beyond the anchoring point 9, runs around the sole again and traverses the middle of the instep in the region of the middle foot 5 until the strap 8 finally reaches the anchoring point 9 where its end will be attached to the strap 8 by means of a hook-and-loop fastener 12 indicated by crosshatching.

This results in a double loop of the strap 8 around the middle foot and furthermore a loop around the lower leg 3 in the region above the ankle in the manner of a figure of eight, which thus ensures high stability of the foot and in particular the ankle joint.

FIG. 2 shows a variant of the embodiment of the ankle joint bandage of FIG. 1, in which the starting point of the strap 8 is identical to that of FIG. 1. In this case, however, the end of the strap, after looping around the middle foot 5 twice, is directed toward the loop 11 of the strap 8 and then attached to the strap 8 at point 14 in the region where it surrounds the lower leg, using a hook-and-loop fastener 13.

This latter way of attaching the strap 8 may likewise make the ankle joint bandage easier to apply. Furthermore, this type of attachment avoids a superimposition of the beginning and the end of the strap which might otherwise lead to an undesired thick portion. From a therapeutic point of view, however, there is no major difference between the ankle joint bandages shown in FIG. 1 and FIG. 2, respectively.

As already mentioned above, the strap may be composed of an inelastic and an elastic portion. This is illustrated in FIG. 1 and FIG. 2 in that the portion of the strap 8 immediately following the anchoring point 9 has parallel longitudinal lines, which is to indicate elasticity. As can be seen from the drawings, this arrangement ends after the strap has looped around the sole side of the middle foot 5 once. Then, from the strap region 10 onwards, the longitudinal hatching is no longer there, meaning that the strap 8 is inelastic in this region.

1. An ankle joint bandage including a stocking (4) which extends at least across the middle foot and the ankle joint, and a fixing strap surrounding the foot (1) wherein the fixing strap, in the form of an elastic strap (8), is medially connected to an anchoring point (9) on the stocking (4) in the middle foot region, from where the strap (8) may then be passed around the middle foot on the sole side and, after crossing the middle of the instep, then loops around the lower leg (3) above the ankle from the medial side, then laterally crosses the middle of the instep again and runs around the middle foot on the sole side once more and may be fixed to a bandage region above the sole.

2. The ankle joint bandage of claim 1 wherein the strap (8), after running around the middle foot on the sole side again, is passed to the region of the starting point (9) by means of a hook-and-loop fastener (12).

3. The ankle joint bandage of claim 1 wherein the strap (8), after running around the middle foot on the sole side again, is passed to the medial side of the strap portion which loops around
the lower leg (3) above the ankle and is fixed to the strap (8) there by means of a hook-and-loop fastener (12).

4. The ankle joint bandage of claim 1 wherein the strap consists of a non-elastic and an elastic portion, with the elastic portion facing the medial starting point (9) and the non-elastic portion facing the hook-and-loop fastener (12; 13).

5. The ankle joint bandage of claim 2 wherein the strap consists of a non-elastic and an elastic portion, with the elastic portion facing the medial starting point (9) and the non-elastic portion facing the hook-and-loop fastener (12; 13).

6. The ankle joint bandage of claim 3 wherein the strap consists of a non-elastic and an elastic portion, with the elastic portion facing the medial starting point (9) and the non-elastic portion facing the hook-and-loop fastener (12; 13).
